(19) United States
(12) Patent Application Publication
(10) Pub. No.: US 2016/0279417 A1
(43) Pub. Date: Sep. 29, 2016

(54) METHODS AND SYSTEMS FOR IMPROVING SPEECH RECOGNITION

(71) Applicant: The Board of Regents of the University of Texas System, Austin, TX (US)

(72) Inventors: Michael P. Kilgard, Richardson, TX (US); Robert L. Rennaker, II, Sachse, TX (US)

(73) Assignee: The Board of Regents of the University of Texas System, Austin, TX (US)

(21) Appl. No.: 14/994,033

(22) Filed: Jan. 12, 2016

Related U.S. Application Data
(60) Provisional application No. 62/102,443, filed on Jan. 12, 2015.

(51) Int. Cl. A61N 1/36; A61N 1/05; A61N 1/04; G10L 15/06; G09B 5/04

(52) U.S. Cl. CPC A61N 1/36053; G10L 15/063; G09B 5/04; A61N 1/0456; A61N 1/0504; A61N 1/36014

(57) ABSTRACT

A method for improving speech recognition, involving assessing a subject's speech recognition ability, selecting a set of speech sounds appropriate for speech recognition training, providing a paired training therapy, and repeating the paired training therapy. The paired training therapy involves selecting a speech sound from the set of speech sounds and introducing said speech sound while concurrently stimulating the subject's vagus nerve.

### CROSS-REFERENCE TO RELATED APPLICATION

The present application claims benefit of and priority to U.S. Provisional Patent Application Ser. No. 62/102,443 filed on Jan. 12, 2015 entitled “Methods and Systems for Therapy for Improving Speech Recognition,” the entire contents of which are hereby incorporated by reference herein.

### BACKGROUND

This invention relates to the field of therapy and rehabilitation for speech impairment.

Neurons in auditory cortex are selective to the spectral and temporal features of environmental sounds. The tuning properties of these neurons can be altered by a variety of conditions.

Deep brain stimulation or cranial nerve stimulation paired with the presentation of a sound can enhance the primary auditory cortex (A1) response to the paired sound. For example, repeated pairing of a tone with stimulation of nucleus basalis or locus coeruleus results in A1 frequency map plasticity that is specific to the paired tone. Pairing vagus nerve stimulation (VNS) with a tone also dramatically increases the percentage of A1 that responds to the paired tone. Pairing stimulation of the nucleus basalis or the vagus nerve with either slow or fast trains of tones either decreases or increases the temporal following rate of A1 neurons.

Auditory system plasticity accelerates auditory learning and could benefit patients with speech and hearing disorders. Many studies have demonstrated that language impaired individuals have weak auditory cortex responses to sound that can be strengthened following extensive rehabilitation therapy. Vagus nerve stimulation is a safe, well-tolerated procedure that is frequently used to treat patients with epilepsy or depression. Pairing VNS with rehabilitation improves recovery from stroke in animal models. Pairing VNS with tones has recently been shown to improve tinnitus symptoms in patients and animal models with chronic tinnitus.

Pairing VNS with tones has recently been shown to improve tinnitus symptoms in both animals with tinnitus and tinnitus patients, with additional clinical trials now underway (clinicaltrials.gov #NCT01962558). Pairing VNS with rehabilitation therapy has improved upper limb function in stroke animals, and studies are ongoing to evaluate the effectiveness of VNS paired with rehabilitation in stroke patients (clinicaltrials.gov identifier NCT01669161 & NCT02243020).

Many patient populations, such as individuals with aphasia (1 million people in the US [NINDS]), deaf individuals (500,000 people in the US), and individuals with autism spectrum disorders (1 in 68 children in the US), suffer from language deficits due to impaired cortical responses to sounds. These individuals require extensive interventions in order to improve speech perception and cortical responses to sounds. For example, auditory cortex responses are slow and weak in deaf individuals. Cochlear implantation and speech therapy improve both cortical responses and speech perception outcomes; however, this process can take many months. Pharmacologically enhanced therapy improves both speech outcomes and auditory cortex responses. Individuals with autism spectrum disorders, particularly those with fragile X syndrome or Rett syndrome, have severe language deficits and impaired cortical responses to sound. Many of these individuals also have epilepsy, and may already have a VNS implant to control their seizures. Pairing speech therapy with VNS could potentially be used to enhance auditory cortex responses and speech perception outcomes in individuals with receptive language deficits.

Tone has one frequency and thus activates a narrow region of the cochlea. Therefore, each tone fires a small proportion of neurons in the auditory system. Speech sounds, on the hand, are broadband stimuli that activate very complex and unpredictable patterns of activity in the central auditory system. One example is a consonant discrimination study in rats wherein “[c]onsonants differing only in their place of articulation resulted in different spatial activity patterns [in the central auditory system].” (Engineer et al 2008) Other studies highlighting the added complexity of speech sounds are listed below and incorporated herein by reference: Kilgard et al 2001; Engineer et al 2008 including its supplementary materials and its supplement; O'Connor et al 2010; Nelken et al 1994; Bar-Yosef et al 2001.

### BRIEF SUMMARY OF THE INVENTION

The patent or application file contains at least one drawing executed in color. Copies of this patent or patent application publication with color drawing(s) will be provided by the Office upon request and payment of the necessary fee.

Accordingly, one example aspect of the present invention is a method for improving speech recognition. The method involves assessing a subject's speech recognition ability, selecting a set of speech sounds appropriate for speech recognition training, providing a paired training therapy, and repeating the paired training therapy. The paired training therapy involves selecting a speech sound from the set of speech sounds and introducing said speech sound while concurrently stimulating the subject's vagus nerve.

Another example aspect of the present invention is a system for improving speech recognition. The system comprises:

assessing a subject's speech recognition ability, wherein assessing the subject's speech recognition ability includes identifying one or more deficiencies in the subject's speech recognition ability;
selecting a set of speech sounds suitable to correct at least one of the deficiencies in the subject's speech recognition ability, the set of speech sounds comprising two or more subsets of the set of speech sounds;
conducting a paired training therapy trial comprising concurrently introducing to the subject a subset of speech sounds and stimulating the subject's vagus nerve, wherein the subset of speech sounds is selected from the two or more subsets of speech sounds and wherein each of the two or more subsets of speech sounds is selected from a group consisting of syllables, words, phrases, and sentences; and
repeating the paired training therapy trial at least 2 times for each of the subsets of speech sounds.

### BRIEF DESCRIPTION OF THE DRAWINGS

FIG. 1 shows a method for improving speech recognition, in accordance with one embodiment of the invention.

FIG. 2 shows the response of VNS paired rats to VNS paired speech sounds (‘lad’ and ‘rad’) and novel speech sounds (‘sad and dad’).

FIG. 3 shows mean spike counts for the responses of control and VNS speech paired rats to VNS paired speech sounds ‘rad’ and ‘lad’.

FIGS. 4A-4B show the latency of primary auditory cortex (A1) responses to paired speech sounds in control and VNS speech paired rats.

FIG. 5 shows the response of control and VNS speech paired rats to tones.

FIGS. 6A-6C show spectrograms, amplitude envelopes, and power spectrums for the paired speech sounds ‘rad’ and ‘lad’.

FIGS. 7A-7B show the primary auditory cortex responses control and VNS speech paired rats in relation to tone frequency and intensity.

FIG. 7C shows the difference in the percent of primary auditory cortex neurons that respond to tones between VNS speech paired rats and control rats.

FIGS. 8A-8B show the number of spikes evoked in control and VNS speech paired rats as a response to tone frequency and intensity.

FIG. 8C shows the difference in the number of spikes evoked between VNS speech paired rats and control rats.

FIGS. 9A-9D shows the onset response of control and VNS speech paired rats to each of the speech sounds (‘rad’, ‘lad’, ‘dad’, and ‘sad’) across characteristic frequencies.

FIG. 10 shows neural detection accuracy for the paired speech sounds ‘rad’ and ‘lad’ and the novel speech sounds ‘dad’ and ‘sad’.

### DETAILED DESCRIPTION OF THE INVENTION

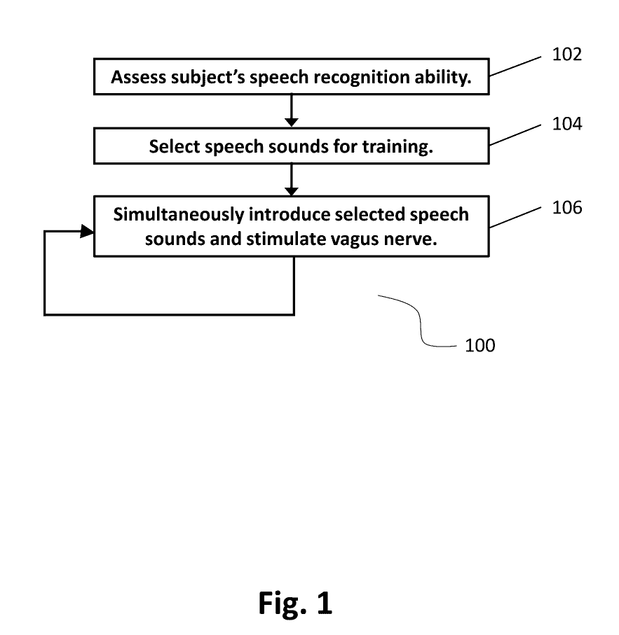

As shown in FIG. 1, one aspect of the present invention is a method 100 for improving speech recognition. The method begins with assessing a subject's speech recognition ability 102. Assessing the subject's speech recognition ability also involves identifying one or more deficiencies in the subject's speech recognition ability.

According to an embodiment of the invention, speech recognition ability appropriate for the instant method may include extreme levels of speech recognition impairment such as speech recognition impairment associated with aphasia and autism. In another embodiment, the instant method 100 may also be appropriate for more moderate levels of speech recognition impairment, such as speech recognition impairment associated with dyslexia.

According to an embodiment of the invention, the subject may be a mammalian subject such as, for example, a human patient.

The method 100 proceeds to selecting a set of speech sounds 104 suitable to correct at least one of the deficiencies in the subject's speech recognition ability. The set of speech sounds comprise two or more subsets of the set of speech sounds. One of ordinary skill in the art would be capable of selecting speech sounds appropriate for addressing these deficiencies.

Once the speech sounds are selected 104, the method 100 proceeds to providing a paired training therapy 106. The paired training therapy involves selecting a speech sound from the set of speech sounds and introducing it while concurrently stimulating the patient's vagus nerve.

The subset of speech sounds is selected from the two or more subsets of speech sounds. Each of the two or more subsets of speech sounds may be selected from a group consisting of syllables, words, phrases, and sentences.

According to an embodiment of the invention, stimulating the subject's vagus nerve may involve applying an electric pulse train using a subcutaneous device.

According to another embodiment of the invention, stimulating the subject's vagus nerve involves using at least one device selected from a group consisting of a subcutaneous device and a transcutaneous device. The subcutaneous and/or the transcutaneous device may stimulate the vagus nerve using electric pulses, magnetic pulses, as well as electric, thermal, light-based, and/or and mechanical activation.

According to an embodiment of the invention, stimulating the subject's vagus nerve may involve applying an electric pulse train using a subcutaneous device. The electric pulse train may have a current amplitude of 0.1 to 2.0 milliamps and a duration of 400 to 600 milliseconds, a current amplitude of 0.2 to 1.0 milliamps and a duration of 400 to 600 milliseconds, a current amplitude of 0.7 to 0.9 milliamps and a duration of 400 to 600 milliseconds, or a current amplitude of 0.3 to 0.5 milliamps and a duration of 400 to 600 milliseconds.

According to an embodiment of the invention, stimulating the vagus nerve may be initiated prior to introducing the selected speech sound, wherein stimulating the vagus nerve may precede introducing the selected speech sound by between 200 ms to 5 ms, or between 100 ms to 20 ms, preferably between 60 ms and 40 ms, such as 50 ms.

According to another embodiment of the invention, the set of speech sounds appropriate for speech recognition training may include similar speech sounds, such as rhyming words, including “lad” and “rad”.

According to an embodiment of the invention, the paired training therapy trial may also be repeated one or more times, such as at least 100 times for each of the subsets of speech sounds.

### Examples

The following embodiments exemplify the methods and systems of the current invention.

### Materials and Methods

### Vagus Nerve Surgery

Sprague Dawley rats were implanted with a custom made platinum iridium bipolar cuff electrode around the left cervical vagus nerve, as in our previous studies. Rats were anesthetized with pentobarbital (50 mg/kg), and received supplemental doses of dilute pentobarbital (8 mg/mL) as needed. Body temperature was maintained at 37° C. using a heating pad, and rats received subcutaneous injections of dextrose and Ringer's lactate for hydration, cefotaxime sodium to prevent infection, and atropine and dexamethasone to decrease bronchial secretions. Leads from the vagus nerve cuff electrode were tunneled subcutaneously to a headcap attached to the skull. Based on previous studies showing no difference between naïve rats and rats that either had implants which were not activated or rats that received VNS which was not paired with any particular event, the control rats in the current study did not undergo sham surgery.

### Speech Sounds

The paired speech sounds were the words ‘rad’ and ‘lad’ spoken by a female native English speaker, as used in our previous studies. The sounds ‘rad’ and ‘lad’ were chosen because they are known to weakly activate A1 neurons, and are known to be perceptually difficult sounds to learn. These characteristics make our results more relevant to conditions, such as dyslexia and autism, which exhibit weak responses to speech sounds that generate strong responses in typically developing individuals.

All sounds were presented so that the loudest 100 ms of the vowel was 60 dB SPL, and the onset of the initial consonant was approximately 40 dB SPL. The sounds were spectrally shifted up by one octave using the STRAIGHT vocoder to better match the rat hearing range.

### Vagus Nerve Stimulation

The words ‘rad’ and ‘lad’ were paired with vagus nerve stimulation 300 times per day for 20 days. The onset of vagus nerve stimulation was 50 ms before the onset of the speech sound. In previous studies, plasticity was indistinguishable when stimulation was 200 ms before sound onset through 50 ms after sound onset. The stimulation burst was a brief 500 ms long pulse train (30 Hz) with a 100 has biphasic pulse width at an intensity of 0.8 mA, as in our previous studies. The amount of VNS used in this study is less than 1% of the FDA approved VNS protocol for epilepsy and depression. The speech sounds were delivered to the unrestrained rats free-field via a speaker (Optimus Bullet Horn Tweeter) located 20 cm above a 25×25×25 cm³ wire cage. Presentation of the speech sounds was randomly interleaved throughout each VNS speech pairing session, and there was no significant difference between the number of times per session that each rat heard ‘rad’ (147±6) compared to ‘lad’ (144±10, p=0.75). The timing of each VNS-speech pairing trial was also randomized so that the rats could not predict when VNS-speech pairing would occur, with an average of 30 seconds between VNS-speech pairing trials. The control rats in this study did not undergo unpaired stimulation. Our previous studies and those of other labs have shown that sound presentation alone (without VNS stimulation) or VNS stimulation alone (without sound presentation) does not substantially alter A1 responses.

### Physiology

Primary auditory cortex (A1) recordings were obtained from each rat 24 hours after the last VNS pairing session, as in our previous studies. Auditory cortex responses were recorded from 263 A1 sites in 4 VNS speech paired rats and 536 A1 sites in 11 control rats. Similar to the vagus nerve surgery, rats were anesthetized with pentobarbital, and supplemental doses of dilute pentobarbital were provided throughout the experiment. Humidified air was delivered through a tracheotomy in order to facilitate breathing. To prevent brain swelling, a cisternal drain was performed. Right primary auditory cortex was exposed following a craniotomy and durotomy. Four Parylene-coated tungsten microelectrodes (1-2 MΩ, FHC) were used to record A1 responses, and were placed to evenly sample A1 while avoiding blood vessels. 1,296 tones were presented at each frequency and intensity combination between 1-32 kHz in 0.125 octave steps and 0-75 dB SPL in 5 dB steps. The paired speech sounds ‘rad’ and ‘lad’ and the novel speech sounds ‘dad’ and ‘sad’ were randomly interleaved twenty times each at every recording site. The novel sounds ‘dad’ and ‘sad’ were spoken by the same female native English speaker and were presented to determine whether plasticity was specific to the paired sounds. Sounds were presented using a speaker located 10 cm from the left ear of the rat.

### Data Analysis

For all analyses, A1 responses in VNS speech paired rats were compared with A1 responses in control rats. A1 recording sites were defined based on latency, tonotopy, and relative location. The onset response strength to speech sounds was the number of evoked spikes fired during the first 40 ms of the response. Previously published research has demonstrated that both humans and animals can reliably discriminate between consonant sounds using only the first tens of milliseconds of the sound. The response strength to the vowel was quantified as the number of spikes evoked during the 300 ms immediately following vowel onset. The peak latency to speech sounds was the latency (in ms) with the maximum firing rate, while the onset latency variance was the square of the standard deviation of the onset latency response to the paired sounds across driven A1 recording sites (in ms2). Neural detection accuracy was calculated using a nearest-neighbor classifier, where 50% correct is chance performance and 100% correct is perfect neural detection. Euclidean distance was used to compare the 40 ms onset response (40 1-ms bins) evoked by each of the speech sounds (′dad′, ‘lad’, ‘rad’, and ‘sad’) with spontaneous firing recorded when no sound was presented. At each A1 recording site, an average sound template post stimulus time histogram (PSTH) was created from 19 of the 20 repeats recorded with and without sound presentation. The PSTH templates were compared to the remaining repeat using Euclidean distance, and each single trial response was assigned to the most similar responding PSTH template with the smallest Euclidean distance. Significance was determined using two-sample t-tests, using a Bonferroni correction for multiple comparisons.

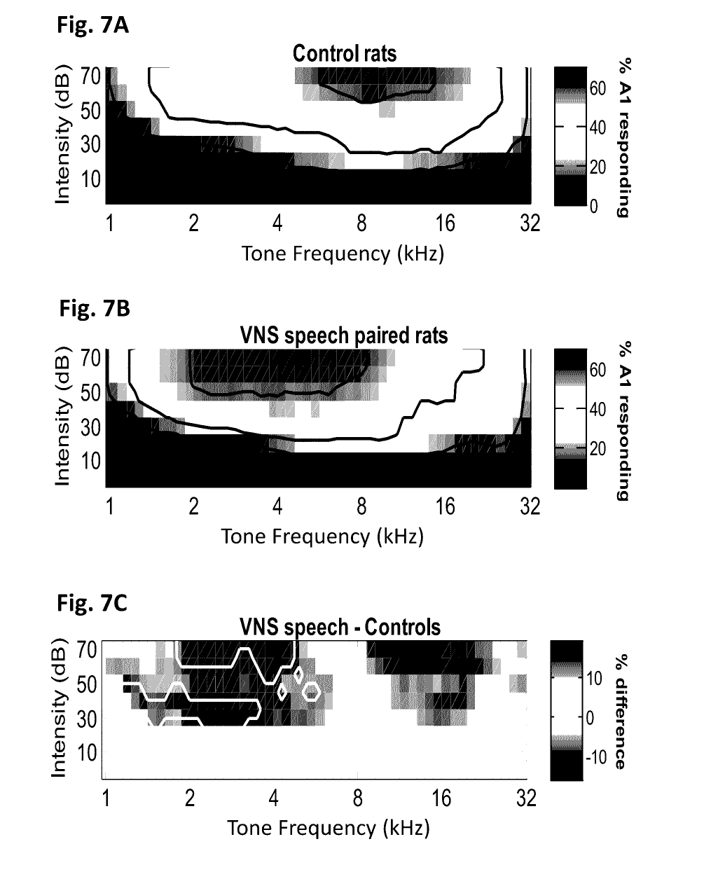

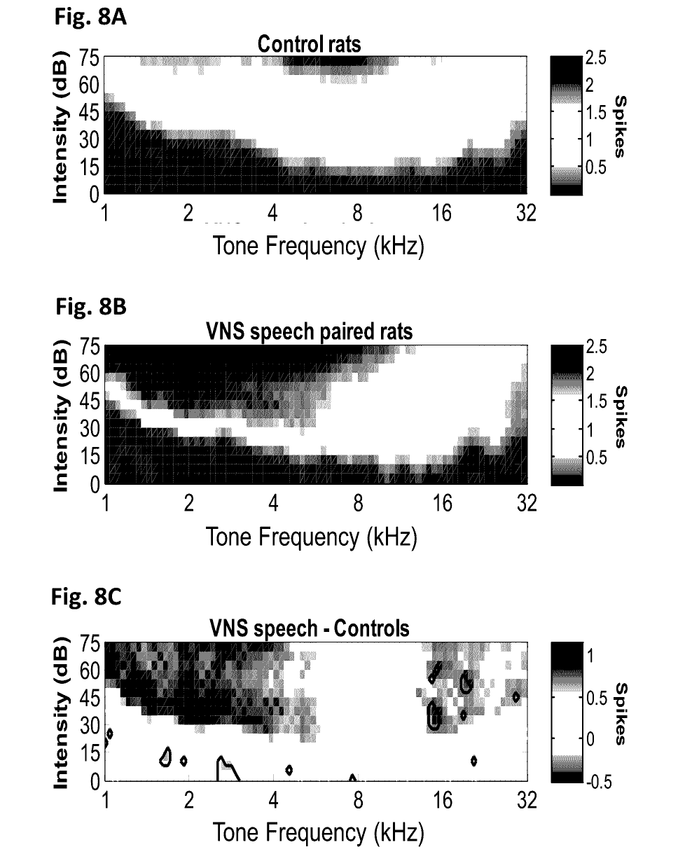

Threshold was the lowest intensity (in dB SPL) that evoked a response at the characteristic frequency for each recording site. Bandwidth was measured 40 dB above each site's threshold as the frequency range (in octaves) that evoked a response. Driven rate was the average response (in spikes/tone) to all of the tones in each site's receptive field. The percent of A1 neurons responding and the number of spikes evoked per tone was calculated for each tone frequency at each tone intensity. For FIGS. 7 and 8, a Benjamini-Hochberg correction was used to control the false discovery rate.

### VNS Pairing Enhanced A1 Responses to the Paired Speech Sounds

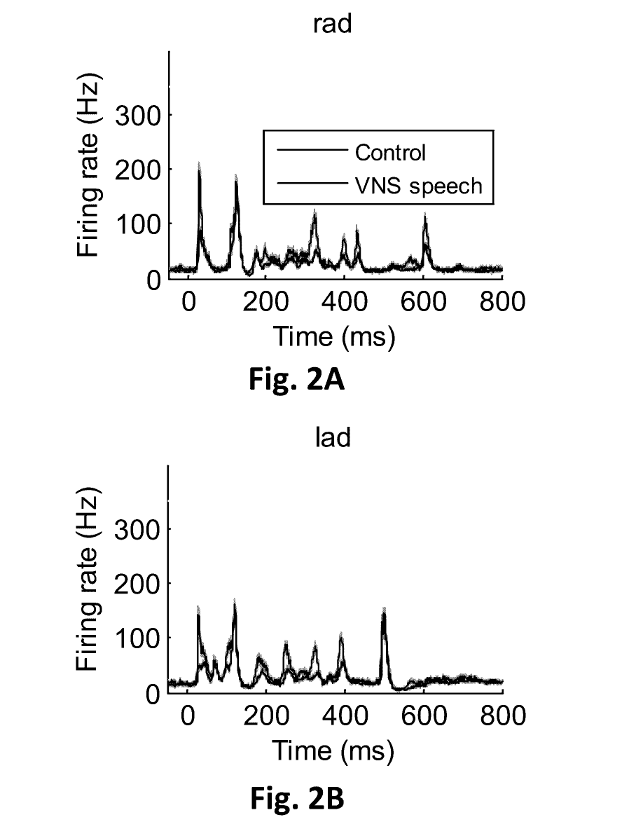

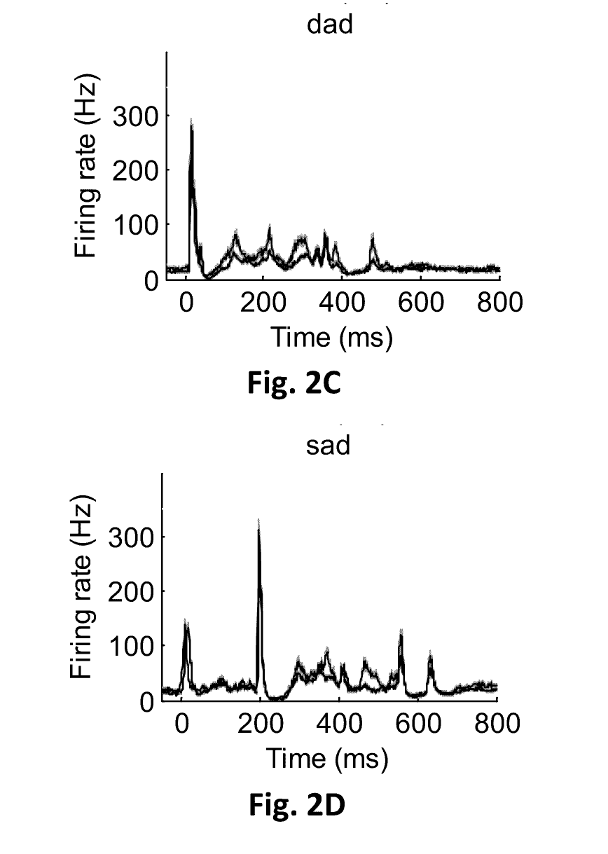

FIGS. 1A-1D show the response of VNS paired rats to VNS paired speech sounds (‘lad’ and ‘rad’) and novel speech sounds (‘sad and dad’). In particular, FIGS. 1A-1D show that VNS speech pairing strengthened the response strength to the paired speech sounds. As shown in FIG. 2A, the mean number of spikes evoked across recording sites in response to the paired speech sound ‘rad’ was significantly stronger in VNS speech paired rats compared to control rats. Gray shading behind each group indicates SEM across recording sites. The waveform for the speech sound ‘rad’ is plotted in gray above the response. As shown in FIG. 2B, the number of spikes evoked in response to the paired speech sound ‘lad’ was significantly stronger in VNS speech paired rats compared to control rats. As shown in FIG. 2C, while the number of spikes evoked in the 40 ms onset response to the novel speech sound ‘dad’ was not significantly different between VNS speech paired and control rats, the VNS speech paired rats exhibited a stronger response to the vowel portion of the sound. As shown in FIG. 2D, while the number of spikes evoked in the 40 ms onset response to the novel speech sound ‘sad’ was significantly weaker in VNS speech paired rats compared to control rats, the VNS speech paired rats exhibited a stronger response to the vowel portion of the sound.

FIG. 3 shows mean spike counts for the responses of control and VNS speech paired rats to VNS paired speech sounds ‘rad’ and ‘lad’. The mean spike count in response to the VNS paired speech sounds ‘rad’ and ‘lad’ was significantly increased in VNS speech paired rats compared to control rats. The driven number of spikes was calculated for each speech sound using the 40 ms onset response to each sound. Error bars indicate SEM across recording sites; asterisks indicate speech sounds with response strengths that were significantly different between VNS speech paired and control rats (p<0.05).

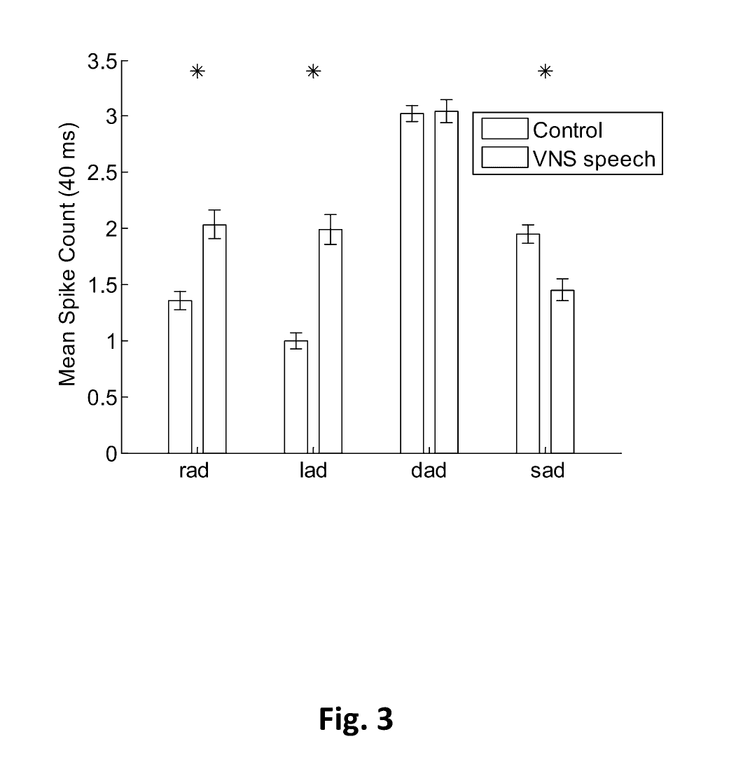

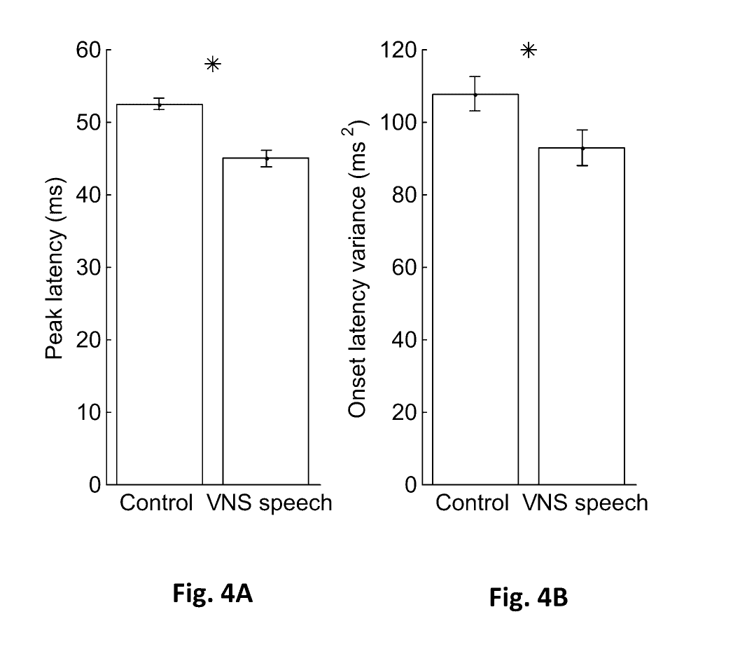

FIGS. 4A-4B show the latency of primary auditory cortex (A1) responses to paired speech sounds in control and VNS speech paired rats. A1 responses to the paired speech sounds were significantly faster in VNS speech paired rats. As shown in FIG. 4A, the peak latency was significantly shorter in VNS speech paired rats compared to control rats. Error bars indicate SEM across recording sites; asterisks indicate a statistically significant difference between VNS speech paired and control rats (p<0.05). As shown in FIG. 4B, the trial-by-trial variability in onset latency was significantly decreased in VNS speech paired rats compared to control rats.

VNS paired with the speech sounds ‘rad’ and ‘lad’ significantly enhanced the A1 response strength to the paired sounds (FIG. 2). Following 20 days of VNS-speech pairing, rats had a 50% stronger onset response to ‘rad’ and a 99% stronger onset response to ‘lad’ compared to control rats (p<0.0001, average number of spikes fired in the first 40 ms of the neural response, FIG. 3). Interestingly, this response strength enhancement did not generalize to novel speech sounds. For example, the onset response strength to the novel sound ‘dad’ did not significantly change in VNS speech rats, while the onset response strength to the novel sound ‘sad’ was actually 26% weaker in VNS speech rats compared to control rats (p=0.0002, average number of spikes fired in the first 40 ms of the neural response, FIG. 3). This pattern of response strength enhancement for the paired sounds but not the novel sounds was observed across a wide range of analysis durations for the consonant response (120 ms for ‘rad’, 110 ms for ‘lad’, 30 ms for ‘dad’, and 210 ms for ‘sad’; each in 10 ms increments). The vowel /ae/ was common across the four speech sounds, and the response strength to the vowel was stronger for both paired and novel speech sounds. The response strength to the vowel in ‘rad’ increased from 4.0±0.2 (mean±SEM) spikes in control rats to 6.9±0.4 spikes in VNS speech paired rats (300 ms vowel response, p<0.0001), the vowel response to ‘lad’ increased from 4.0±0.2 spikes in controls to 6.3±0.5 spikes in VNS speech paired rats (p<0.0001), the vowel response to ‘dad’ increased from 4.6±0.2 spikes to 7.2±0.5 spikes in VNS speech paired rats (p<0.0001), and the vowel response to ‘sad’ increased from 4.6±0.2 spikes to 6.0±0.4 spikes in VNS speech paired rats (p=0.0002, FIG. 2). This stronger response strength to the vowel in VNS speech paired rats was observed across a wide range of analysis durations for the vowel response (200-500 ms in 100 ms increments, p<0.05).

In addition to stronger A1 responses to the paired speech sounds, the A1 responses to the paired sounds were also faster. The peak firing latency to the paired sounds was significantly faster in VNS speech rats compared to control rats (44.9±1.2 ms vs. 52.4±0.8 ms, p<0.0001, FIG. 4A). A1 neurons also fired more reliably to the paired sounds in VNS speech rats compared to control rats (latency variance of 92.7±4.9 ms2 vs. 107.7±4.7 ms2, p=0.03, FIG. 4B). The peak firing latency to the novel sounds was unaltered in VNS speech rats compared to control rats (23.5±1.1 ms vs. 23.7±0.6 ms, p=0.84). In contrast to the paired sounds, A1 neurons fired less reliably to novel sounds in VNS speech rats compared to control rats (latency variance of 36.4±2.8 ms2 vs. 27.6±1.7 ms2, p=0.004).

### VNS Pairing Alters A1 Receptive Fields

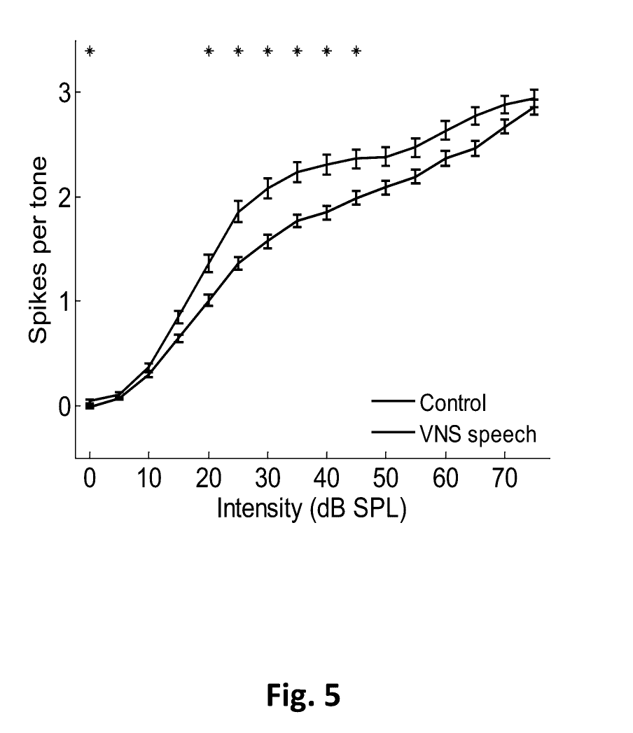

FIG. 5 shows the response of control and VNS speech paired rats to tones. As shown in FIG. 5, the response strength to tones was significantly stronger in VNS speech paired rats. VNS speech paired rats evoked more spikes per tone at intensities between 20-45 dB SPL compared to control rats. Responses are the average spikes evoked per tone for tones within 1 octave of each A1 recording site's characteristic frequency. Asterisks indicate intensities that evoke a stronger response in VNS speech paired rats compared to control rats (p<0.0031, Bonferroni correction). Error bars indicate SEM across recording sites.

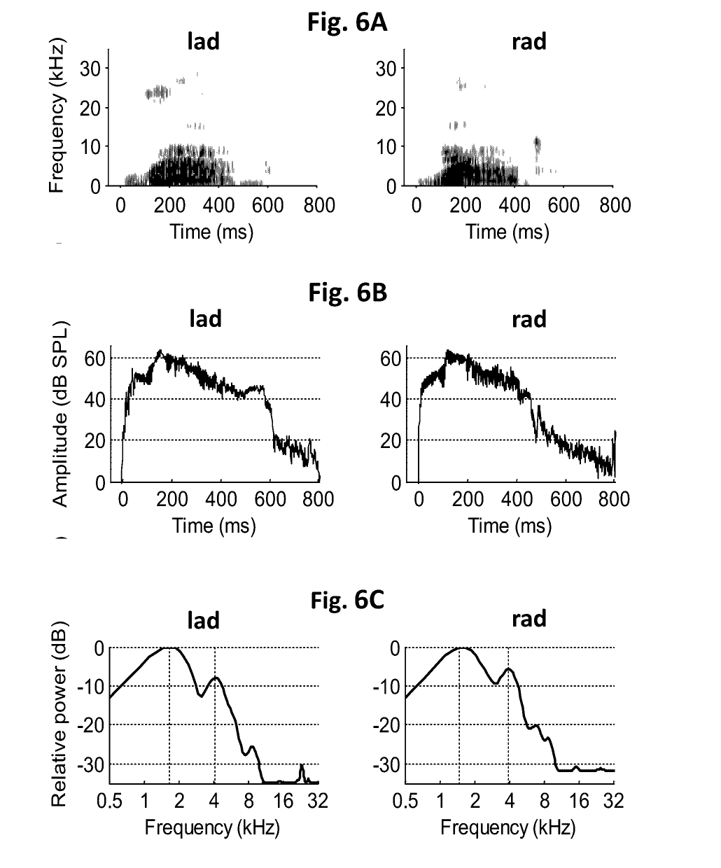

FIGS. 6A-6C show spectrograms, amplitude envelopes, and power spectrums for the paired speech sounds ‘rad’ and ‘lad’. FIG. 6A shows the spectrogram for the paired sounds ‘rad’ and ‘lad’. Time is represented on the x axis (−50 to 800 ms), and frequency is represented on the x axis (0 to 35 kHz). The intensity of the sound is plotted so that white is 70 dB SPL quieter than black. FIG. 6B shows the amplitude envelopes for ‘rad’ and ‘lad’ and FIG. 6C shows the power spectrums for ‘rad’ and ‘lad’.

FIGS. 7A-7B show the primary auditory cortex responses to tone frequency and intensity of control and VNS speech paired rats. The percentage of primary auditory cortex responding to low frequency tones increased in VNS speech paired rats. FIG. 7A shows the percent of primary auditory cortex neurons that respond to a tone of any frequency and intensity combination in control rats. Black contour lines indicate 20, 40, and 60% of primary auditory cortex responding. FIG. 7B shows the percent of primary auditory cortex neurons that respond to tones in VNS speech paired rats. FIG. 6C shows the difference in the percent of primary auditory cortex neurons that respond to tones between VNS speech paired rats and control rats. White contour lines surround the regions of tones that were significantly different compared to control rats (false discovery rate was used to correct for multiple comparisons).

FIGS. 8A-8B show the number of spikes evoked in control and VNS speech paired rats as a response to tone frequency and intensity. The number of spikes evoked per tone in VNS speech paired rats increased for low frequency tones and decreased for high frequency tones. FIG. 8A shows the number of spikes evoked in response to any frequency and intensity combination of tones in control rats. FIG. 8B shows the number of spikes evoked in response to any frequency and intensity combination of tones in VNS speech paired rats. FIG. 8C shows the difference in the number of spikes evoked between VNS speech paired rats and control rats. White contour lines surround the tone regions that were significantly increased (false discovery rate was used to correct for multiple comparisons) compared to control rats, while black contour lines surround the tone regions that were significantly decreased compared to control rats.

VNS speech pairing significantly altered primary auditory cortex responses to tones. A1 neurons were able to respond to tones that were 3.3 dB quieter in VNS speech paired rats compared to control rats (p<0.0001, Table 1). These paired neurons were able to respond to frequencies spanning an additional 0.2 octaves compared to control neurons (p=0.009, Table 1). VNS speech paired responses to tones were 1.1 ms faster (p=0.01) and 0.4 spikes per tone stronger (p=0.001) compared to responses in control rats (Table 1). A1 responses to tones were significantly stronger (p<0.0031, FIG. 5) in VNS speech paired rats compared to control rats at tone intensities ranging from 20-45 dB SPL, which matches the intensity of the initial consonants in the paired sounds ‘rad’ and ‘lad’ (FIG. 6). A1 responses to tones were both stronger and faster following VNS speech pairing, and VNS speech paired neurons responded to quieter tones and a wider range of frequencies compared to control neurons.

TABLE 1
VNS speech pairing induced receptive field plasticity

| | Control | VNS speech | p value |
|---|---|---|---|
| Threshold (dB) | 18.61 | 15.27 | p < 0.0001 |
| Bandwidth 40 | 2.54 | 2.72 | p = 0.009 |
| (octaves) | | | |
| Peak latency (ms) | 19.95 | 18.88 | p = 0.01 |
| Driven rate | 3.07 | 3.47 | p = 0.001 |
| (spikes/tone) | | | |

Previous studies have demonstrated that vagus nerve or nucleus basalis stimulation paired with a tone increases the percent of A1 that responds to the paired tone. Since the paired speech sounds ‘rad’ and ‘lad’ are low frequency biased sounds (FIG. 6), it is possible that the stronger response strength to the paired speech sounds is simply due to an expansion of the percentage of A1 that responds to low frequency sounds. We quantified the percent of cortex responding to tones with all frequency and intensity combinations to determine if VNS speech pairing results in A1 frequency map plasticity. At 60 dB SPL, approximately 16% more A1 neurons responded to frequencies between 1.9-4.9 kHz in VNS speech paired rats compared to control rats (p<0.05, FIG. 7).

The number of spikes evoked per tone was then quantified to determine whether VNS speech paired rats have both more neurons that respond to low frequency sounds, as well as stronger responses to low frequency sounds. At 60 dB SPL, A1 neurons responded on average 50% stronger to low frequency tones below 6 kHz in VNS speech paired rats compared to control rats (p<0.05, FIG. 8).

The Low Frequency Map Expansion does not Fully Account for the Enhanced Speech Responses

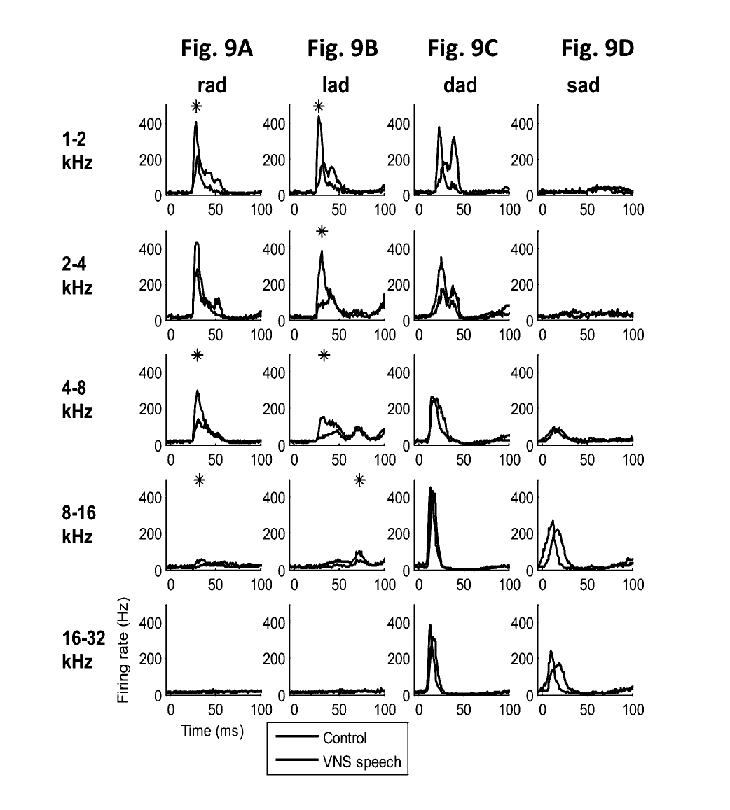

FIGS. 9A-9D shows the onset response of control and VNS speech paired rats to each of the speech sounds (‘rad’, ‘lad’, ‘dad’, and ‘sad’) across characteristic frequencies. The paired sounds ‘rad’ (FIG. 9A) and ‘lad’ (FIG. 9B) evoked a strong response in low frequency tuned neurons. The novel sound ‘dad’ (FIG. 9C) evoked a response across all frequency ranges, while the novel sound ‘sad’ (FIG. 9D) evoked a response in high frequency tuned neurons. Asterisks indicate a significantly stronger peak firing rate across recording sites in VNS speech paired rats compared to control rats (Bonferroni correction for multiple comparisons, p<0.01).

The enhanced A1 response strength to the paired speech sounds was not fully explained by the increased low frequency map representation. Low frequency neurons responded with a higher peak firing rate to the paired speech sounds ‘rad’ and ‘lad’ in VNS speech paired rats compared to control rats (p<0.01, FIGS. 8A, 8B). Even A1 sites tuned to tone frequencies above 6 kHz exhibited a stronger peak firing rate to the paired speech sounds in VNS speech paired rats compared to control rats (p<0.01, FIG. 8a,b). In contrast, the peak firing amplitude to the novel speech sounds ‘dad’ and ‘sad’ was not significantly altered in VNS speech paired rats compared to control rats (p>0.05, FIG. 8c,d). The latency to peak response was decreased for both paired and novel speech sounds in VNS speech paired rats compared to control rats (p<0.01, FIG. 9).

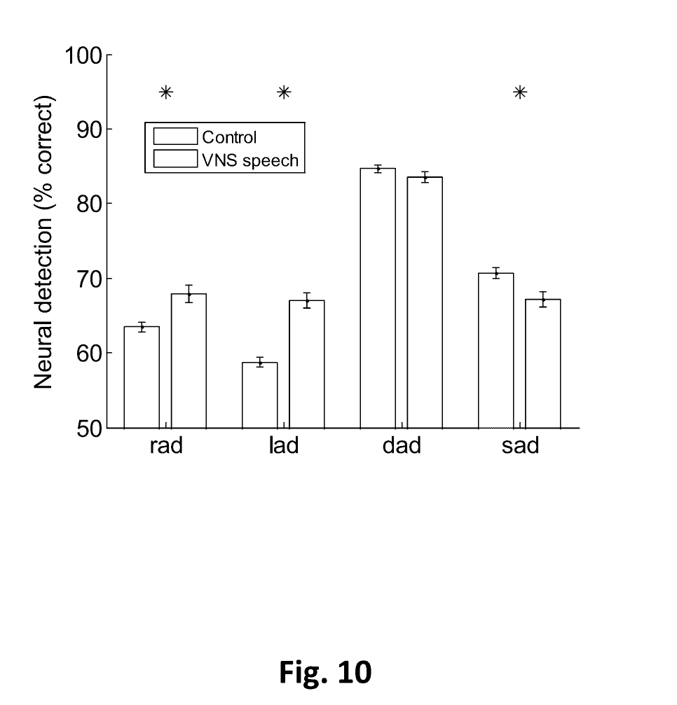

FIG. 10 shows neural detection accuracy for the paired speech sounds ‘rad’ and ‘lad’ and the novel speech sounds ‘dad’ and ‘sad’. Neural detection performance significantly increases for the paired speech sounds ‘rad’ and ‘lad’, does not change for the novel speech sound ‘dad’, and significantly decreases for the novel speech sound ‘sad’. Error bars indicate SEM across recording sites; asterisks indicate a statistically significant difference between VNS speech paired and control rats (p<0.05).

The stronger response strength, faster latency, and decreased latency variance of the evoked responses to the paired speech sounds improved the ability of a neural classifier to detect the onset of the paired sounds. Neural detection of the two paired sounds was significantly more accurate in VNS speech paired rats compared to control rats across all A1 recording sites (p<0.001, FIG. 10). Neural detection of the novel speech sound ‘dad’ was not altered (p=0.22), while neural detection of the novel speech sound ‘sad’ was 4% less accurate in VNS speech paired rats compared to control rats (p=0.005, FIG. 10). This finding suggests that VNS speech pairing can increase the neural detection accuracy of the paired sounds by making A1 responses stronger, faster, and more reliable.

Many studies document auditory cortex plasticity specific to the acoustic characteristics of the presented sounds. In this study, we extend these findings by showing that VNS paired with speech sounds enhanced the A1 response to the paired speech sounds. The A1 response evoked by the paired sounds ‘rad’ and ‘lad’ was stronger, faster and less variable following 20 days of VNS speech pairing. In contrast, the amplitude of the response evoked by novel speech sounds was not strengthened. A1 receptive fields were altered, and this plasticity was specific to the frequency and intensity characteristics of the paired sounds. A neural classifier was significantly more accurate at detecting the paired speech sounds, while neural detection accuracy was not enhanced for novel speech sounds.

### Receptive Field Plasticity

The speech sounds used in this example were low frequency sounds with most of their energy below 4 kHz. Following VNS speech pairing, the A1 representation of low frequency sounds was expanded and these low frequency tuned neurons were also stronger. This finding is consistent with previous studies that paired tones with stimulation of the nucleus basalis or vagus nerve. Extensive speech sound discrimination training also results in an expansion and strengthening of the low frequency A1 response.

In this example, the speech sounds were presented so that the loudest portion of the vowel was 60 dB SPL, while the initial consonants were between 20 and 45 dB SPL. Following VNS speech pairing, the A1 response to these middle intensities was stronger, which matches the intensity of the paired consonants. This finding matches previous findings showing intensity specific plasticity following tone intensity training.

Previous studies have documented that pairing a tone with nucleus basalis stimulation can increase receptive field size and decrease latency. Each of the receptive field changes observed in this study are consistent with previously documented A1 changes following tone pairing or tone training.

### Speech Sound Plasticity

This study has documented A1 plasticity that is specific to the acoustic characteristics of the paired sounds. The stronger response strength evoked by the paired speech sounds in this study was not restricted to the low frequency map expansion. High frequency neurons responded stronger to both of the paired speech sounds after VNS speech pairing. These neurons did not respond more strongly to the novel speech sounds, but did decrease their latency to peak amplitude for the novel speech sounds. The high frequency tuned neurons had increased receptive field sizes (Table 1) so that they were able to respond to lower frequency sounds, such as the paired sounds ‘rad’ and ‘lad’, following VNS speech pairing. Neural detection ability of the paired speech sounds increased, while the neural detection ability of novel speech sounds was not enhanced.

### REFERENCES

[1] Kilgard M P, Merzenich M M. Distributed representation of spectral and temporal information in rat primary auditory cortex. Hear Res 1999; 134:16-28.
[2] Buonomano D V, Merzenich M M. Cortical plasticity: from synapses to maps. Annu Rev Neurosci 1998; 21:149-86.
[3] Bakin J S, Weinberger N M. Induction of a physiological memory in the cerebral cortex by stimulation of the nucleus basalis. Proc Natl Acad Sci USA 1996; 93:11219-24.
[4] Kilgard M P, Merzenich M M. Cortical Map Reorganization Enabled by Nucleus Basalis Activity. Science (80-) 1998; 279:1714-8.
[5] Engineer N D, Riley J R, Seale J D, Vrana W A, Shetake J A, Sudanagunta S P, et al. Reversing pathological neural activity using targeted plasticity. Nature 2011; 470:101-4.
[6] Kilgard M P, Merzenich M M. Plasticity of temporal information processing in the primary auditory cortex. Nat Neurosci 1998; 1:727-31.
[7] Edeline J-M, Manunta Y, Hennevin E. Induction of selective plasticity in the frequency tuning of auditory cortex and auditory thalamus neurons by locus coeruleus stimulation. Hear Res 2011; 274:75-84.
[8] Shetake J A, Engineer N D, Vrana W A, Wolf J T, Kilgard M P. Pairing tone trains with vagus nerve stimulation induces temporal plasticity in auditory cortex. Exp Neurol 2012; 233:342-9.
[9] Kilgard M P, Merzenich M M. Order-sensitive plasticity in adult primary auditory cortex. Proc Natl Acad Sci USA 2002; 99:3205-9.
[10] Moucha R, Pandya P K, Engineer N D, Rathbun D L, Kilgard M P. Background sounds contribute to spectrotemporal plasticity in primary auditory cortex. Exp Brain Res 2005; 162:417-27.
[11] Pandya P K. Speech sound representation and experience-dependent plasticity in the rat auditory system. The University of Texas at Dallas, 2005.
[12] Hays S A, Rennaker R L, Kilgard M P. Targeting plasticity with vagus nerve stimulation to treat neurological disease. Prog Brain Res 2013; 207:275-99.
[13] Reed A C, Riley J, Carraway R S, Carrasco A, Perez C A, Jakkamsetti V, et al. Cortical map plasticity improves learning but is not necessary for improved performance. Neuron 2011; 70:121-31.
[14] Kilgard M P. Harnessing plasticity to understand learning and treat disease. Trends Neurosci 2012.
[15] De Ridder D, Vanneste S, Engineer N D, Kilgard M P. Safety and efficacy of vagus nerve stimulation paired with tones for the treatment of tinnitus: a case series. Neuromodulation 2014; 17:170-9.
[16] Dawson G, Jones E J H , Merkle K, Venema K, Lowy R, Faja S, et al. Early behavioral intervention is associated with normalized brain activity in young children with autism. J Am Acad Child Adolesc Psychiatry 2012; 51:1150-9.
[17] Dawson G, Rogers S, Munson J, Smith M, Winter J, Greenson J, et al. Randomized, controlled trial of an intervention for toddlers with autism: the Early Start Denver Model. Pediatrics 2010; 125:e17-23.
[18] Fu Q-J, Galvin J J. Maximizing cochlear implant patients' performance with advanced speech training procedures. Hear Res 2008; 242:198-208.
[19] Vollmer M, Beitel R E. Behavioral training restores temporal processing in auditory cortex of long-deaf cats. J Neurophysiol 2011; 106:2423-36.
[20] Englot D J, Chang E F, Auguste K I. Vagus nerve stimulation for epilepsy: a meta-analysis of efficacy and predictors of response. J Neurosurg 2011; 115:1248-55.
[21] Sackeim H A, Rush A J, George M S, Marangell L B, Husain M M, Nahas Z, et al. Vagus nerve stimulation (VNS) for treatment-resistant depression: efficacy, side effects, and predictors of outcome. Neuropsychopharmacology 2001; 25:713-28.
[22] Clark K B, Naritoku D K, Smith D C, Browning R a, Jensen R a. Enhanced recognition memory following vagus nerve stimulation in human subjects. Nat Neurosci 1999; 2:94-8.
[23] Khodaparast N, Hays S A, Sloan A M, Hulsey D R, Ruiz A, Pantoja M, et al. Vagus nerve stimulation during rehabilitative training improves forelimb strength following ischemic stroke. Neurobiol Dis 2013; 60:80-8.
[24] Khodaparast N, Hays S A, Sloan A M, Fayyaz T, Hulsey D R, Rennaker R L, et al. Vagus Nerve Stimulation Delivered During Motor Rehabilitation Improves Recovery in a Rat Model of Stroke. Neurorehabil Neural Repair 2014.
[25] Engineer C T, Perez C A, Chen Y H, Carraway R S, Reed A C, Shetake J A, et al. Cortical activity patterns predict speech discrimination ability. Nat Neurosci 2008; 11:603-8.
[26] Engineer C T, Perez C A, Carraway R S, Chang K Q, Roland J L, Kilgard M P. Speech training alters tone frequency tuning in rat primary auditory cortex. Behav Brain Res 2014; 258:166-78.
[27] Logan J S, Lively S E, Pisoni D B. Training Japanese listeners to identify English/r/ and /l/: a first report. J Acoust Soc Am 1991; 89:874.
[28] Kawahara H. Speech representation and transformation using adaptive interpolation of weighted spectrum: Vocoder revisited. Proc ICASSP 1997; 2:1303-6.
[29] Porter B A, Khodaparast N, Fayyaz T, Cheung R J, Ahmed S S, Vrana W A, et al. Repeatedly pairing vagus nerve stimulation with a movement reorganizes primary motor cortex. Cereb Cortex 2012; 22:2365-74.
[30] Ranasinghe K G, Carraway R S, Borland M S, Moreno N A, Hanacik E A, Miller R S, et al. Speech discrimination after early exposure to pulsed-noise or speech. Hear Res 2012; 289:1-12.
[31] Yan J, Zhang Y. Sound-guided shaping of the receptive field in the mouse auditory cortex by basal forebrain activation. Eur J Neurosci 2005; 21:563-76.
[32] Ma X, Suga N. Augmentation of plasticity of the central auditory system by the basal forebrain and/or somatosensory cortex. J Neurophysiol 2003; 89:90-103.
[33] Polley D B, Read H L, Storace D A, Merzenich M M. Multiparametric auditory receptive field organization across five cortical fields in the albino rat. J Neurophysiol 2007; 97:3621-38.
[34] Centanni T M, Engineer C T, Kilgard M P. Cortical speech-evoked response patterns in multiple auditory fields are correlated with behavioral discrimination ability. J Neurophysiol 2013; 110:177-89.
[35] Blumstein S E, Stevens K N. Acoustic invariance in speech production: evidence from measurements of the spectral characteristics of stop consonants. J Acoust Soc Am 1979; 66:1001-17.
[36] Bertoncini J, Bijeljac-Babic R, Blumstein S E, Mehler J. Discrimination in neonates of very short CVs. J Acoust Soc Am 1987; 82:31-7.
[37] Porter B A, Rosenthal T R, Ranasinghe K G, Kilgard M P. Discrimination of brief speech sounds is impaired in rats with auditory cortex lesions. Behav Brain Res 2011; 219:68-74.
[38] Perez C A, Engineer C T, Jakkamsetti V, Carraway R S, Perry M S, Kilgard M P. Different timescales for the neural coding of consonant and vowel sounds. Cereb Cortex 2013; 23:670-83.
[39] Benjamini Y, Hochberg Y. Controlling the false discovery rate: a practical and powerful approach to multiple testing. J R Stat Soc Ser B . . . 1995; 57:289-300.
[40] Polley D B, Steinberg E E, Merzenich M M. Perceptual learning directs auditory cortical map reorganization through top-down influences. J Neurosci 2006; 26:4970-82.
[41] Kilgard M P, Pandya P K, Vazquez J, Gehi A, Schreiner C E, Merzenich M M. Sensory input directs spatial and temporal plasticity in primary auditory cortex. J Neurophysiol 2001; 86:326-38.
[42] Blanchfield B B, Feldman J J, Dunbar J L, Gardner E N. The severely to profoundly hearing-impaired population in the United States: prevalence estimates and demographics. J Am Acad Audiol 2001; 12:183-9.
[43] Prevalence of autism spectrum disorder among children aged 8 years—autism and developmental disabilities monitoring network, 11 sites, United States, 2010. MMWR Surveill Summ 2014; 63:1-21.
[44] Nittrouer S, Sansom E, Low K, Rice C, Caldwell-Tarr A. Language Structures Used by Kindergartners With Cochlear Implants: Relationship to Phonological Awareness, Lexical Knowledge and Hearing Loss. Ear Hear 2014:1-13.
[45] Moore D R, Shannon R V. Beyond cochlear implants: awakening the deafened brain. Nat Neurosci 2009; 12:686-91.
[46] Lai G, Schneider H D, Schwarzenberger J C, Hirsch J. Speech stimulation during functional M R imaging as a potential indicator of autism. Radiology 2011; 260:521-30.
[47] Callan D E, Tajima K, Callan A M, Kubo R, Masaki S, Akahane-Yamada R. Learning-induced neural plasticity associated with improved identification performance after training of a difficult second-language phonetic contrast. Neuroimage 2003; 19:113-24.
[48] Naito Y, Hirano S, Honjo I, Okazawa H, Ishizu K, Takahashi H, et al. Sound-induced activation of auditory cortices in cochlear implant users with post- and prelingual deafness demonstrated by positron emission tomography. Acta Otolaryngol 1997; 117:490-6.
[49] Sharma A, Dorman M F, Spahr A J. A sensitive period for the development of the central auditory system in children with cochlear implants: implications for age of implantation. Ear Hear 2002; 23:532-9.
[50] Oh S-H, Kim C-S, Kang E J, Lee D S, Lee H J, Chang S O, et al. Speech perception after cochlear implantation over a 4-year time period. Acta Otolaryngol 2003; 123:148-53.
[51] Sharma A, Gilley P M, Dorman M F, Baldwin R. Deprivation-induced cortical reorganization in children with cochlear implants. Int J Audiol 2007; 46:494-9.
[52] Tobey E A, Devous M D, Buckley K, Overson G, Harris T, Ringe W, et al. Pharmacological enhancement of aural habilitation in adult cochlear implant users. Ear Hear 2005; 26:455-56S.
[53] Stach B A, Stoner W R, Smith S L, Jerger J F. Auditory evoked potentials in Rett syndrome. J Am Acad Audiol 1994; 5:226-30.
[54] Van der Molen M J W, Van der Molen M W, Ridderinkhof K R, Hamel B C J, Curfs L M G, Ramakers G J a. Auditory change detection in fragile X syndrome males: a brain potential study. Clin Neurophysiol 2012; 123:1309-18.
[55] Levy M L, Levy K M, Hoff D, Amar A P, Park M S, Conklin J M, et al. Vagus nerve stimulation therapy in patients with autism spectrum disorder and intractable epilepsy: results from the vagus nerve stimulation therapy patient outcome registry. J Neurosurg Pediatr 2010; 5:595-602.
[56] Wilfong A a, Schultz R J. Vagus nerve stimulation for treatment of epilepsy in Rett syndrome. Dev Med Child Neurol 2006; 48:683-6.
[57] Hays S A, Khodaparast N, Hulsey D R, Ruiz A, Sloan A M, Rennaker R L, et al. Vagus nerve stimulation during rehabilitative training improves functional recovery after intracerebral hemorrhage. Stroke 2014; 45:3097-100.
[58] Bar-Yosef O, Rotman Y, Nelken I. Responses of Neurons in Cat Primary Auditory Cortex to Bird Chirps: Effects of Temporal and Spectral Context. J Neurosci 2002; 22:8619-32.
[59] O'Connor K N, Ying P, Petkov C I, Sutter M L. Complex Spectral Interactions Encoded by Auditory Cortical Neurons: Relationship Between Bandwidth and Pattern. Front Syst Neurosci. 2010; 4:145.

What is claimed is:

1. A method for improving speech recognition, the method comprising:
assessing a subject's speech recognition ability, wherein assessing the subject's speech recognition ability includes identifying one or more deficiencies in the subject's speech recognition ability;
selecting a set of speech sounds suitable to correct at least one of the deficiencies in the subject's speech recognition ability, the set of speech sounds comprising two or more subsets of the set of speech sounds;
conducting a paired training therapy trial comprising concurrently introducing to the subject a subset of speech sounds and stimulating the subject's vagus nerve, wherein the subset of speech sounds is selected from the two or more subsets of speech sounds.

2. The method of claim 1, wherein stimulating the patient's vagus nerve involves applying an electric pulse train using a subcutaneous device.

3. The method of claim 1, wherein stimulating the subject's vagus nerve involves using at least one device selected from a group consisting of a subcutaneous device and a transcutaneous device.

4. The method of claim 1, further comprising repeating the paired training therapy trial one or more times.

5. The method of claim 1, further comprising repeating the paired training therapy trial at least 100 times for each of the subsets of speech sounds.

6. The method of claim 4, further comprising starting stimulation of the vagus nerve between 200 ms to 5 ms prior to introducing the subset of speech sounds.

7. The method of claim 4, further comprising starting stimulation of the vagus nerve between 100 ms to 20 ms prior to introducing the subset of speech sounds.

8. The method of claim 4, further comprising starting stimulation of the vagus nerve 60 ms to 40 ms prior to introducing the subset of speech sounds.

9. The method of claim 2, wherein the electric pulse train includes an electric pulse with a current amplitude of 0.1 to 2.0 milliamps and a duration of 400 to 600 milliseconds.

10. The method of claim 2, wherein the electric pulse train includes an electric pulse with a current amplitude of 0.2 to 1.0 milliamps and a duration of 400 to 600 milliseconds.

11. The method of claim 2, wherein the electric pulse train includes an electric pulse with a current amplitude of 0.7 to 0.9 milliamps and a duration of 400 to 600 milliseconds.

12. The method of claim 2, wherein each of the two or more subsets of speech sounds is selected from a group consisting of syllables, words, phrases, and sentences.

13. The method of claim 2, wherein the electric pulse train includes an electric pulse with a current amplitude of 0.3 to 0.5 milliamps and a duration of 400 to 600 milliseconds.

14. The method of claim 4, wherein the set of speech sounds appropriate for speech recognition training comprises similar speech sounds.

15. The method of claim 1, wherein the similar speech sounds include “lad” and “rad”.

16. A system for improving speech recognition, the system comprising:
assessing a subject's speech recognition ability, wherein assessing the subject's speech recognition ability includes identifying one or more deficiencies in the subject's speech recognition ability;
selecting a set of speech sounds suitable to correct at least one of the deficiencies in the subject's speech recognition ability, the set of speech sounds comprising two or more subsets of the set of speech sounds;
conducting a paired training therapy trial comprising concurrently introducing to the subject a subset of speech sounds and stimulating the subject's vagus nerve, wherein the subset of speech sounds is selected from the two or more subsets of speech sounds and wherein each of the two or more subsets of speech sounds is selected from a group consisting of syllables, words, phrases, and sentences; and
repeating the paired training therapy trial at least 2 times for each of the subsets of speech sounds.

17. The system of claim 16, wherein stimulating the patient's vagus nerve involves using a device selected from a group consisting of a subcutaneous device and a transcutaneous device.

18. The system of claim 16, further comprising starting stimulation of the vagus nerve between 100 ms to 20 ms prior to introducing the subset of speech sounds.

19. The system of claim 16, further comprising starting stimulation of the vagus nerve 60 ms to 40 ms prior to introducing the subset of speech sounds.

20. The system of claim 17, wherein the electric pulse train includes an electric pulse with a current amplitude of 0.2 to 1.0 milliamps and a duration of 400 to 600 milliseconds.
